(19) United States
(12) Patent Application Publication
(10) Pub. No.: US 2006/0110833 A1
(43) Pub. Date: May 25, 2006

(54) Method and apparatus for coupling an analyte supply to an electrodynamic droplet processor

(76) Inventors: George R. Agnes, Coquitlam (CA); Michael J. Bogan, Burnaby (CA)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 11/223,456

(22) Filed: Sep. 9, 2005

Related U.S. Application Data
(60) Provisional application No. 60/608,083, filed on Sep. 9, 2004.

(51) Int. Cl. G01N 33/00; B01D 59/44; H01J 49/00

(52) U.S. Cl. 436/86; 250/282

(57) ABSTRACT

This application relates to a method and apparatus for coupling an analyte supply, such as a biomolecule separator, to an electrodynamic droplet processor. In one embodiment the biomolecule separator is a capillary liquid chromatography column and the droplet processor includes an droplet generator and an electrodynamic balance. The biomolecule separator and the droplet processor may be fluidly coupled to provide a continuous supply of analyte for analysis. The droplets may be controllably ejected from the electrodynamic balance and deposited on a target substrate for use in detecting the analyte by mass spectrometry, such as MALDI time of flight mass spectrometry. Prior to deposition, each of the droplets is levitated in the electrodynamic balance for a period sufficient to enable evaporation of volatile solvents present in the droplet solution, thereby increasing the analyte concentration in the droplet. The solution may include a MALDI liquid matrix and the target substrate may be a MALDI plate. In one embodiment, the method involves depositing a succession of discrete droplets on the target substrate to form one or more microspots having a high density of analytes. The microspots are then irradiated and the ions produced are analyzed by mass spectrometry. The invention improves the sensitivity of analyte detection while consuming a comparatively small volume of test solution.

### REFERENCE TO RELATED APPLICATION

This application claims the benefit of U.S. provisional patent application Ser. No. 60/608,083 filed 9 Sep. 2004 which is hereby incorporated by reference.

### TECHNICAL FIELD

This application relates to a method and apparatus for coupling an analyte supply, such as a biomolecule separator, to an electrodynamic droplet processor. The droplets may be controllably deposited on a target substrate for use in detecting the analyte by mass spectrometry, such as MALDI time of flight mass spectrometry.

### BACKGROUND

MALDI and LDI are methods of producing ions from sample material. The term “MALDI” refers to “matrix assisted laser desorption/ionization”. The term “LDI” refers to “laser desorption/ionization”. The most common way of detecting the ions produced by these ion sources is by mass spectrometry. [1-3] Thus the ion sources (MALDI and LDI) are commonly integrated with a mass spectrometer (MS). The most common type of mass spectrometer used in this application is a time of flight (TOF) mass spectrometer. Such-an ion generation and detection process is therefore sometimes referred to as MALDI-TOF-MS. This process is described in detail in the Applicant's prior international application No. PCT/CA01/01496 filed 23 Oct. 2001 and entitled “Method and Apparatus for Producing a Discrete Particle” (WO 2002/035553 A3) and PCT/CA2004/000242 filed 24 Feb. 2004 (WO 2004/075208 A3) and entitled “Formation of Closely Packed Microspots and Irradiation of Same”, the disclosures of which are hereby incorporated herein by reference.

The MALDI source is sometimes referred to in the mass spectrometry literature as a “soft” ionization source. The term “soft” implies that this ion source allows for the detection of intact compounds, even though the compounds are considered fragile (i.e. the compounds easily decompose with the addition of energy). An example of a common MALDI-TOF-MS application is the detection of peptides generated by proteolytic digestion of proteins in a sample, or proteins, oligossacharrides, RNA, DNA and other polymeric materials. [4-7] The MALDI technique may also be effective in analyzing other large biomolecules. One reason that MALDI has become a very successful and widely used technique for preparing gas-phase ions of biomolecules for mass spectrometry is that the preparation of discrete crystallized sample spots is amenable to high-throughput automated analyses.

The MALDI ion source involves the irradiation of a sample using a pulsed laser that causes the desorption/ ionization of molecules in the sample spot. [8] Irradiated samples can be in a solid or liquid form, though solid samples are more commonly encountered. Conventionally, a solid sample is prepared by mixing an aliquot of the sample with an aliquot of a matrix solution, then the mixture is delivered (i.e. pipetted) onto a substrate and volatile solvents are allowed to evaporate, leaving behind a solid residue that contains the non-volatile species from the sample plus matrix compound(s). It is believed that the MALDI source results in little fragmentation of the analyte compounds because the technique involves the use of a matrix that is mixed with the sample at a mole ratio of ˜1000:1 chromophore:analyte. The matrix is in fact a chromophore that absorbs the output of the pulsed laser used in the MALDI experiment. The matrix absorbs the radiation from the pulsed laser and is itself vaporized and partially decomposed. During the vaporization, analyte molecules are also carried into the gas phase and by either direct ionization or secondary ionization, the analyte molecules become ionized. [9, 10] Direct ionization is the absorption of the laser radiation and ejection of an electron from the analyte. Secondary ionization refers to gas-phase ion-molecule reactions in the plume of material desorbed by the laser. The extent of secondary ionization is not well characterized in the prior art.

The ease with which an analyst prepares a sample for characterization by MALDI is itself easy, simple, and fast: an analyst need only mix the sample with a matrix solution. An aliquot, or all of that mixture is then deposited onto a substrate and the volatile solvent in that mixture is allowed to evaporate dry to leave a dry, solid residue. That residue is then targeted with the laser in the MALDI-TOF-MS instrument. In principle, the preparation of sample material for MALDI-TOF-MS analysis is trivial. In reality, the most frequently encountered problem in the technique is that the sample is simply not detected. There are many reasons for that, such as the threshold level for laser power prior to observing analyte ions. Because of this and other easily and commonly observed characteristics of MALDI, it is widely believed that the detection of an analyte compound in a MALDI experiment critically depends on the co-crystallization of the analyte compounds with the matrix.

The Applicant's prior international applications referred to above (WO 02/035553 A3 and WO 2004/075208 A3) describe electrodynamically levitating a sample particle, which may include a solid member, a droplet, a single molecule, or a cluster of molecules, and delivering the particle to a target location. This process is sometimes referred to as “wall-less sample preparation” (WaSP). Briefly, in one embodiment the WaSP technology involves the use of an ink-jet droplet generator to create droplets from a starting solution. In order to levitate the droplets in the electrodynamic balance (EDB) a net charge is induced on to the droplet. Though other forms of levitation could be used, each would have their own constraints on the physical and chemical composition of the droplet. The volatile solvents in the starting solution, such as methanol and water, rapidly evaporate (i.e. typically within 1-2 seconds) from the droplet. The evaporation of volatile solvents concentrates the non-volatile (plus low volatility) solutes that were in the starting solution inside what is now descriptively referred to as the levitated droplet residue. That droplet residue is then deposited onto a target substrate. Translating the substrate relative to the EDB, or vice versa, and repeating the process of creating and levitating a droplet followed by the deposition of that residue allows a user of WaSP to pattern multiple spots of materials onto a substrate.

MALDI has helped revolutionize the study of biomolecules by mass spectrometry due, in part, to its ability to create primarily singly charged and intact analyte ions from a sample spot composed of, most commonly, a solid matrix within which the analyte was co-crystallized. Liquid matrices developed for UV-MALDI have been shown to have some useful characteristics, such as increased shot-to-shot reproducibility, but are not commonly used because of adduct formation, relatively poor resolution, and higher detection limits [11-16]. Based on their studies of a chemical-doped glycerol matrix that enabled picomole sensitivities for proteins prepared as a 2 μL sample spot, Sze et al. speculated that the use of smaller matrix droplets and delayed ion extraction could improve the analytical utility of matrix solutions [17].

The major challenges in attaining sample spots with small diameters (<100 μm) include the manipulation of sub-nanoliter volumes and the spreading of solution upon deposition. Consequently, several researchers have developed dedicated approaches for sample deposition and/or used pre-structured sample supports including the use of piezoelectric droplet deposition [18-20], heated sample plates [21], filling micromachined picoliter vials using a glass micropipette [22], hydrophilic sample anchors on hydrophobic surfaces [23], picoliter syringes [24], and vacuum deposition of a liquid exiting a capillary [25, 26]. All of these techniques result in the formation of solid matrix sample spots and rely on changing the surroundings into or onto which a droplet or liquid stream was deposited, thus requiring some form of pre-structured sample support, heat source, or a sub-atmospheric pressure chamber.

The present invention pertains to a new strategy for coupling an analyte supply, such as a capillary liquid chromatography column or other biomolecule separator, with off-line MALDI-MS. In particular, WaSP is used as a post-column pre-concentrating interface between the biomolecule separator or other analyte supply and a target MALDI plate.

### SUMMARY OF THE INVENTION

In accordance with the invention a method of preparing samples for use in analyte detection is provided. In-one embodiment, the method comprises providing a supply of analyte; forming a test solution comprising the analyte and at least one volatile solvent; generating a discrete droplet of the solution; electrodynamically levitating the droplet to enable evaporation of the volatile solvent, thereby increasing the concentration of the analyte in the droplet; and controllably depositing the droplet at a target location on a substrate to create at least one microspot thereon. The method may further comprise repeating the droplet generation, levitation and deposition steps to successively deposit multiple droplets at the target location, thereby increasing the density of the analyte in the microspot. The analyte may then be detected by irradiating the microspot and detecting ions produced by the irradiating by mass spectrometry, such as time of flight mass spectrometry.

In one particular embodiment of the invention the analyte may be a biomolecule provided from an upstream biomolecule separator, such as a capillary liquid chromatography column.

The invention also relates to an analyte detection system for implementing the above-described methods. In one embodiment the system comprises an analyte supply; a vessel for forming a test solution comprising the analyte and at least one volatile solvent; a particle generator for generating discrete droplets of the solution; and an electrodynamic balance for electrodynamically levitating the droplets produced by the droplet generator for a sufficient length of time to enable evaporation of the volatile solvent and hence concentration of the analyte in the droplets, wherein the electrodynamic balance successively ejects droplets to a target location following levitation thereof.

Many further embodiments of the invention are described and claimed herein.

### BRIEF DESCRIPTION OF DRAWINGS

In drawings which illustrate embodiments of the invention, but which should not be construed as restricting the spirit or scope of the invention in any way,

FIG. 1 is a schematic view showing a biomolecule separator, namely a capillary liquid chromatography column, operatively coupled to a droplet processor. The figure illustrates the experimental approach to compare the preparation of a capLC fractionated peptide using (A) a dried droplet, (B) a droplet dispenser, and (C) a droplet dispenser in conjunction with electrodynamic WaSP processing. The time elapsed for a single droplet to be dispensed until its impact with the MALDI plate in (B) and (C) is labelled as t₁ and t₂ respectively. The inset of (C) is an artistic impression of the lengthened oscillatory trajectory of the droplets passing through the EDB that causes t₂ to be greater than t₁.

FIG. 2 is an enlarged schematic view of a particle levitation chamber. A micrometer translation stage moves the MALDI plate along the y-axis.

FIG. 3 are light microscopy images from an optical microscope showing CHCA crystals in sample spots each prepared from 1000 droplets dispensed directly onto a stainless steel MALDI target plate at (A) 3 Hz, (b) 10 Hz, and (C) 50 Hz.

FIG. 4 is a schematic view illustrating two modes of operation of wall-less sample preparation (WaSP) using (A) Dynamic electric fields and (B) Static Electric Fields operated with 1 Hz droplet dispensing.

FIG. 5 are light microscopy images from an optical microscope of CHCA crystals in sample spots prepared by WaSP from droplets created with IP_{f}=(A) 90 V or (B) 170 V. (i) 100 droplets, 3 Hz; (ii) 1000 droplets, 3 Hz; (iii) 1000 droplets, 10 Hz; (iv) 1000 droplets, 50 Hz.

FIG. 6 are reflectron mode MALDI mass spectra of the peptide T²²⁶-Y²⁴⁰ collected from a liquid chromatography fraction (40-45 minutes) prepared at three different droplet dispensing speeds using (A) direct deposition onto the target plate from a piezodispenser or wall-less sample preparation of droplets that had been created with (B) IP_{f}=90 V and IP_{f}=170 V.

FIG. 7 are reflectron mode MALDI mass spectra of the peptide T²²⁶-Y²⁴⁰ collected from a liquid chromatography fraction (40-45 minutes) prepared using (A) a 1.00 μL dried droplet (<90 fmol consumed) and (B) 1000 droplets (260 nL, <40 fmol consumed) processed by wall-less sample preparation (IP_{f}=170 V.) Insets depict the isotopic resolution (note y-axis scale on insets).

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

The present invention provides an interface between an analyte supply, such as a biomolecule separator, and an electrodynamic droplet processor. For example, the biomolecule separator may be a capillary liquid chromatography (capLC) column and the droplet processor may be used to prepare a sample for MALDI mass spectrometry. Coupling of liquid separation technologies with mass spectrometry (MS) forms the basis for powerful analytical strategies used to separate and identify biomolecules. [27-35] The present invention enables reliable on-line coupling of liquid separation and mass spectrometry technologies to achieve highly sensitive analyte detection. Coupling of such technologies is well-suited to automation as will be appreciated by a person skilled in the art.

In one particular embodiment, the invention may be employed to detect the presence of analytes present in a test solution in low concentration, such as small amounts of proteins, peptides or other biomolecules. The biomolecules may be derived, for example, from an upstream biomolecule separator. The capLC column is one example of a biomolecule separator widely employed in proteomics research.

CapLC columns typically have inner diameters <500 μm and employ flow rates spanning from 10 μl n down to 10 nl/min. Initial efforts to couple capLC to MS were directed towards micro or nano dimension electrospray ion sources. [36-39] However, the ES ion source is relatively sensitive to impurities and solvent conditions, rendering it incompatible with some liquid chromatography gradient elution conditions. Thus, strategies to enable coupling between capLC and matrix-assisted laser desorption/ ionization (MALDI) have been developed to take advantage of the robust characteristics of this soft ion source. [31, 40-42] Several benefits can be gained by coupling capLC to MALDI including (1) those inherent to MALDI: greater tolerance to impurities, compatibility with TOF mass spectrometers, and spectral simplicity and (2) those resulting from the “archival” of the sample on the MALDI target: temporal constraints of the LC separation are not imposed, facile reanalysis of old separations, opportunity for on-probe manipulations of target analytes (e.g. desalting, dephosphorylation).

In coupling capLC to MALDI in the prior art, the column effluent has been collected offline on a target plate in a continuous streak, [43,44] or as a series of discrete sample spots created from a piezodispenser, [45,46] by ES deposition, [47] through a heated nebulizer [41] or using hanging droplets in a heated interface. [42] Each of these strategies can be adapted to allow online mixing of the matrix with the column effluent prior to deposition or direct deposition of the column effluent onto a pre-prepared matrix surface.

The present invention introduces an alternative strategy for coupling capLC with offline MALDI-MS. As described above, wall-ess sample preparation (WaSP) [48,49] is methodology that utilizes electrodynamic levitation technology [50-52] to control the trajectories of picoliter volume charged droplets dispensed from a droplet-on-demand piezoelectric droplet dispenser. Importantly, the levitated droplets are able to be deposited on a target plate to form sample spots (sometimes referred to herein as “microspots”) with high spatial accuracy which greatly facilitates their subsequent analysis. An important aspect of the processing of the charged droplets during transit to the MALDI target is that volatile solvents contained in the droplets evaporate rapidly [53], effecting non-volatile solute concentration factors in the range 10¹ to 10³. In this application the significant potential of using WaSP as a post-column pre-concentrating interface between capLC and a target plate for off-line matrix-assisted laser desorption/ionization (MALDI) mass spectrometry (MS) is established.

As will be appreciated by a person skilled in the art, many variations and embodiments of the invention are possible. For example, the MALDI matrix may be mixed with the analyte in the test solution which is used for droplet generation or the matrix may be applied to the target substrate, either before or after droplet deposition. Further, although the invention is described herein principally with reference to MALDI-TOF MS, the invention may be employed to couple flow from an analyte supply, such as a biomolecule separator, to any instrumental method of analysis. For example, the invention may be employed to form sample spots for fluorescence detection. In another example, the analyte may be ejected directly to on-line instrumentation, such as an on-line electrospray mass spectrometer, rather than depositing droplets on a target substrate such as a MALDI plate.

The following Example illustrates the invention in further detail although it will be appreciated that the invention is not limited to the specific Example.

### EXAMPLE

During the development of this interface, a sample derived from the limited proteolysis of an amphitropic membrane protein, cytidine 5′-triphosphate:phosphocholine cytidylyltransferase α-isoform (CCT), was studied. CCT is a 367 amino acid membrane protein that regulates phosphatidylcholine synthesis in animal cells, reversibly binding to nuclear membrane lipids in a process that regulates its function. [54] The peptides generated from the limited proteolysis were used as a representative sample to mimic the type of sample encountered in a protein identification experiment.

Recombinant rat CCT was purified from a baculovirus expression system using the method of Friesen et al. [55] as modified by Davies et al. [56] A 60 second reaction period of 3 pmol/μL CCT with α-chymotrypsin using a digestion method that has been described in detail previously, [57] yielded the peptides studied. The reaction was stopped after one minute by adding phenylmethylsulfonyl fluoride which inhibits α-chymotrypsin activity. Capillary liquid chromatographic separation of the peptides was performed using a CapLC System (Waters Technologies, Milford, Mass.) with a Symmetry300 C₁₈, 5 μm packing diameter, 0.32 I. D.×150 mm length column. The following conditions were applied during the run: injection volume 2.0 μl, flow rate 5.00 μL/min, gradient: Solution A=0.1% TFA in ACN, Solution B=H₂O, min, 3% A−97% B,90 min, 43% A−57% B. Fractions were collected in microcentrifuge tubes at 5 minute intervals, 25 μl per fraction, 18 fractions in total.

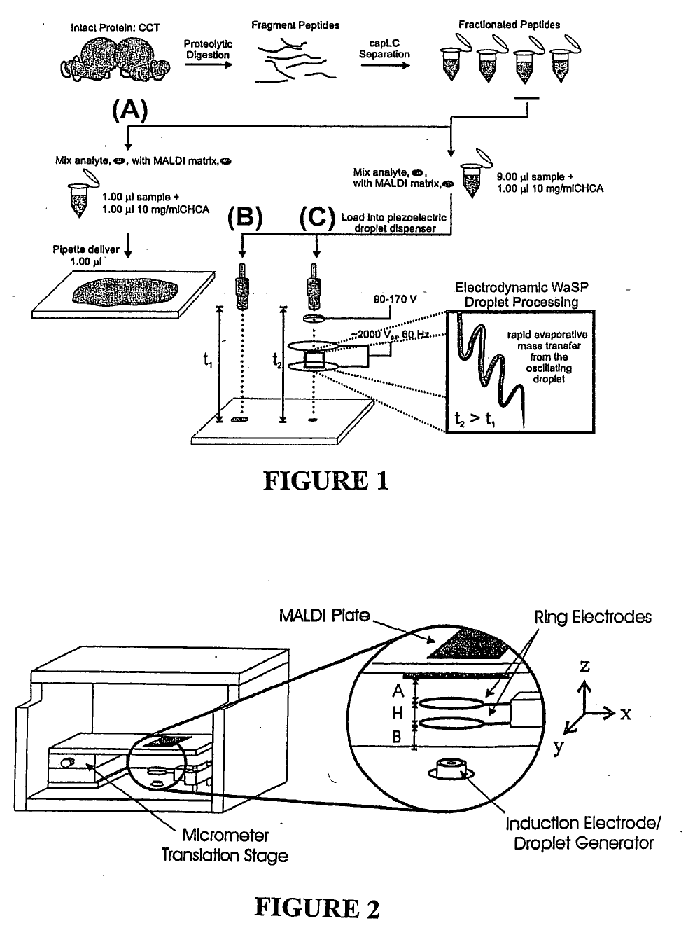

Each fraction was prepared first as a dried droplet as follows (FIG. 1A). A 1.00 μl aliquot of the fraction was added to 1.00 μl of a solution containing 10.0 mg/ml α-cyano-4-hydroxycinnamic acid (CHCA) prepared in 50:50 MeOH:0.1% TFA in acetonitrile. 1.00 μL of this mixture was spotted onto the MALDI target and allowed to dry. The peptides in each fraction were identified using MALDI-TOF-MS (MALDI-LR, Waters Technologies, Milford, Mass.). In linear mode a pulse voltage of 925 V was applied. For reflectron mode, the pulse voltage was 2450 V and the reflectron voltage was 2000 V. In both modes the source voltage was 15000 V and the multichannel plate detector was operated at 1800 V.

The fraction corresponding to 40-45 minutes (fraction 40-45) was singled out for additional analysis. This fraction contained the peptide T²²⁶-Y²⁴⁰ (TAKELNVSFINEKKY, protonated monoisotopic m/z=1783.96). This peptide was also observed in fraction 45-50 minutes at ˜25% the signal intensity of fraction 40-45. A total of 6 pmol CCT (5 μl injection volume) was loaded onto the column. The actual concentration of the peptide was estimated by assuming that 75% of the total amount of peptide was in fraction 40-45. The original 5 μl injection was diluted to 25 μl in the fraction so the concentration of T²²⁶-Y²⁴⁰ in fraction 40-45 would have been ˜180 fmol/μL had the proteolytic digestion been allowed to go to completion. However this was not a complete digestion so this value represents the maximum concentration of the peptide in fraction 40-45. A 1.00 μL aliquot of a 10.0 mg/ml CHCA solution prepared in 50:50 MeOH:0.1% TFA in acetonitrile was added to 9.00 μL fraction 40-45, and ˜5 μL of this solution was loaded into a piezoelectric droplet-on-demand dispenser (Microfab technologies Ltd., Plano, Tex.) fitted with a 40 μm diameter orifice (FIG. 1B). The initial volume of the droplets dispensed were nominally 300 pL. At a distance of ˜2 mm from the MALDI target, 1000 droplets were dispensed at 3, 10, or 50 Hz directly onto the stainless steel target plate. This created three distinct sample spots composed of the co-crystallized matrix and peptide material from 1000 droplets in each spot (FIG. 3).

Using WaSP to couple capLC to MALDI necessitates the capability of operating in a mode that electrodynamically processes and delivers droplets to the MALDI plate with sufficient speed to accommodate the flow rates required for efficient capLC separations. Using the same starting solution as in the piezoelectric droplet dispensing, sample spots composed of the co-crystallized matrix and peptide material were also created by WaSP processing, with each replicate involving 1000 droplets, at 3, 10, and 50 Hz (FIG. 1C). WaSP methodology has been described in detail previously and is shown schematically in FIG. 2. [22, 32] Briefly, an electrode, referred to as the induction electrode, was positioned 3 mm from the orifice of the droplet dispenser. Variation of the DC potential applied to the induction electrode (IP_{f}, the induction potential during droplet formation) proportionally varied the magnitude of the image charge imparted onto the forming droplets. Here, during droplet formation, one of two induction electrode potentials were used: IP_{f}=90 V or IP_{f}=170 V. A double-ring electrodynamic balance (EDB) was used to levitate the charged droplets. A 60 Hz AC potential applied to both ring electrodes provided a radial restoring force and a DC field was used to offset gravity.

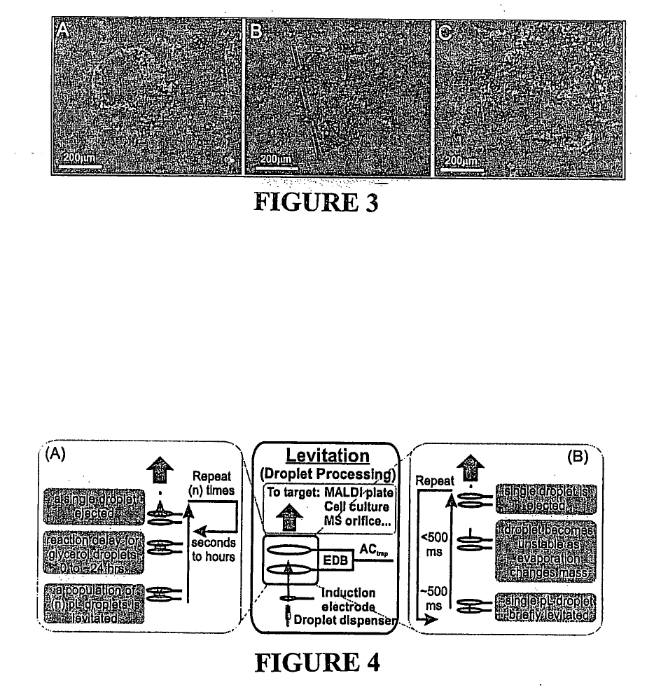

As described above, previous demonstrations of WaSP have focused on the delivery of single droplets at a time, from a population levitated in an EDB, to a remote target. [48, 58] This was achieved by modifying the potential applied to the remote target to attract the droplets out of the EDB (FIG. 4A). Within that methodology, the population of droplets, once levitated, was trapped for a delay time from seconds to hours in length, depending on the rates evaporation from the levitated droplets, before they were deposited. In forming arrays of sample spots from the levitated droplets using this mode, note that only the droplet ejection step was repeated, no new droplets were introduced into the population. With respect to time, the DC potential applied to the remote target was dynamic, either increasing to attract droplets out of the EDB or decreasing to collapse the population back into the EDB. This mode will hereafter be termed “dynamic” mode. Dynamic mode WaSP is generally not amenable to coupling with LC because it is unable to accommodate the flow rates required.

If a flow rate of up to 5 μL/min could be accommodated, gradient elution of peptides or other biomolecules by capLC could be performed routinely. This would require 300 pL droplets to be processed by WaSP at a rate of ˜280 Hz. To approach this rate of droplet delivery through WaSP, a second mode was developed (FIG. 4B). [32] Previously, single droplets were created (IP_{f}=20 V) at a frequency of 1 Hz while the AC field applied to the ring electrodes (ACₜᵣₐₚ)=2700 V with the potential applied to the target plate (DP) was set at 200 V. This caused each droplet to be briefly trapped in the EDB, allowing a majority of the methanol contained in the droplets to evaporate. Within a period of time <1 s, each droplet was injected into the EDB, briefly levitated, and ejected along the z axis at x=y=0. By moving the MALDI plate between each droplet generation event, an array of deposited droplets was formed. Note that when operating in this mode the electric fields were not changed over time. Thus this mode is referred to herein as the “static” or capLC coupling mode.

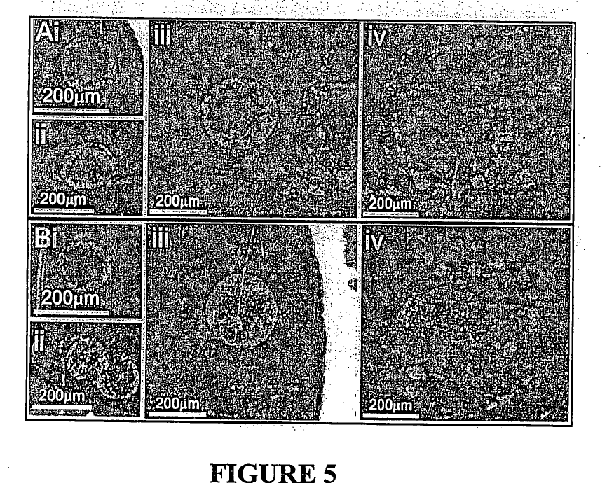

In this Example, the static mode of WaSP operation was modified to create conditions that would simulate coupling to capLC. First droplets were processed with IP_{f}=90 V, DP=1000 V, and ACₜᵣₐₚ=2100 V. Instead of translating the plate between each droplet deposition, multiple droplets were deposited on a single position thereby concentrating the sample. Droplets created from a solution containing <180 fmol/μL of the CCT peptide T²²⁶-Y²⁴⁰ and 1.0 mg/ ml of CHCA matrix were dispensed at 3, 10, and 50 Hz, corresponding to flow rates of 47, 156, and 780 nL/min respectively. FIG. 5A shows light microscopy of the co-crystallized sample spots produced with varied droplet dispensing rates and numbers of droplets deposited. Note that when delivered at 3 Hz, 1000 droplets are contained within the same area as 100 droplets (spot size was ˜190 μm diameter), representing an increase in analyte density of an order of magnitude. When the rate of droplet dispensing was increased to 10 Hz and 50 Hz, the diameter of the sample spot increased to ˜230 μm and ˜600 μm, respectively.

In previous WaSP work, it was shown that the most important factor impacting the proximity with which droplets could be co-deposited was ensuring that only a single droplet was ejected from the EDB at any one time. [48] If more than one droplet was ejected at a time, Coulomb repulsion between droplets being ejected from the EDB caused them to be deposited at off-axis positions of up to 200 μm, which decreased the sample concentration in any one area relative to when only a single droplet was ejected at any one time. To investigate the potential of Coulomb repulsion having an impact when attempting to use WaSP to couple capLC and MALDI, droplets were also processed with IP_{f}=170 V, DP=1000 V, and ACₜᵣₐₚ=1450 V. Note the higher IP_{f} results in a greater induced net charge on each droplet. Droplets were prepared at the same dispensing rates and numbers as for the IP_{f}=90 V droplets. The light microscopy of the sample spots created from these droplets showed subtle differences between the two (compare FIGS. 5A and 5B). For example, for the sample spot prepared by processing 1000 droplets at 3 Hz, it appeared as though some of the droplets were not deposited directly on top of each other so the size of the sample spot cannot be compared to the corresponding IP_{f}=90 V sample spot. Also, when the frequency was increased to 50 Hz, patterns developed in the deposition positions of the WaSP processed droplets created at both IPf's (FIGS. 5Aiv and 4Biv). Specific positions, ˜20-30 μm in diameter within an area of ˜1000 μm in diameter were preferential for droplet deposition. For the lP_{f}=90 V droplets, 9 of these distinct regions were observed, whereas the IP_{f}=170 V droplets created 13-14 distinct regions. Because the droplets were being ejected at 10 ms intervals and the droplets with higher net charge were affected more (FIG. 5Biv), it was likely that Coulomb repulsion between the droplets was influencing their deposition locations as observed previously using dynamic mode WaSP. [22] When the number of droplets was increased to 2000, there were still only 13-14 distinct deposition positions. This suggested that this focusing of droplet trajectories by Coulomb repulsion could provide very large concentration factors of the LC column eluent, forming multiple discrete sample spots while operating at very high droplet dispensing rates. By creating several different sample spots from a single fraction of column eluent, this strategy would enable several different on-probe manipulations to be performed on the same fraction.

Comparison of droplets processed at 10 Hz (FIGS. 3B and 5Aiii, 5Biii), showed that direct droplet dispensing produced a sample spot almost twice as large in diameter as the WaSP processed sample spots (420 μm vs. 230 μm). Because the analyte density is a function of the square of the radius, the analyte concentration in the WaSP processed sample spot was increased by at least 3 times. The cause of the increased sample spot size for direct droplet dispensing is not directly evident from the light microscopy images shown. During droplet deposition onto the target plate, when the droplets were not processed by WaSP, they were observed to accumulate on the target plate to form a larger droplet. The WaSP processed droplets, however, experienced longer trajectories when being delivered to the MALDI plate because they were manipulated by the oscillating electric field of the EDB. This not only increased the time available for evaporation of solvent, but the oscillatory motion of droplets in an EDB increased the rate of solvent evaporation. [53] Thus, the WaSP processed droplets prepared smaller sample spots, and based on the optical images of those spots, they also had higher density crystal formation than those dispensed directly onto the stainless steel MALDI target plate at the same frequencies.

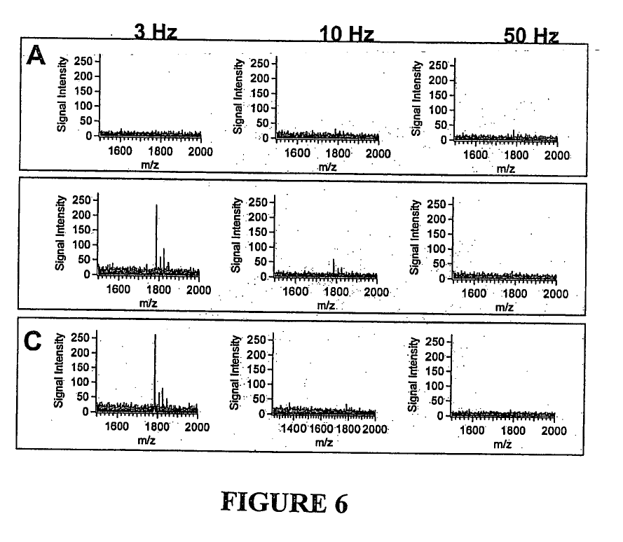

Comparison of the intensity of the [T²²⁶-Y²⁴⁰+H⁺] ion detected by MALDI-MS of the sample spots in FIGS. 3 and 5 revealed that WaSP processing of fraction 40-45 produced higher ion intensities than direct deposition from the droplet dispenser onto the MALDI plate even though the same solution was used in both methods. This was a direct result of the increased analyte concentration produced by preparing smaller sample spots from identical aliquots of a starting solution. FIG. 6 shows that the most efficient sample preparation was achieved by WaSP processing of 1000 droplets at 3 Hz with either IP_{f}=90 V or IP_{f}=170 V. As mentioned earlier, this corresponds to a flow rate of about 47 nL/min.

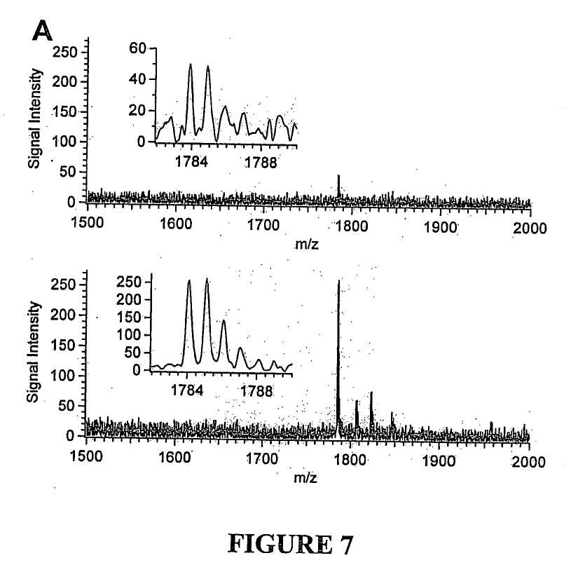

When compared to the dried droplet preparation of fraction 40-45, WaSP processing of droplets created at either IPf produced higher ion intensity (FIG. 7). The Na⁺ and K⁺ adducts detected in FIG. 7B may have arisen because WaSP droplet processing could also concentrate impurities that may be present in the starting solution or introduced by the droplet dispenser. However, they also could have arisen simply because of the higher sensitivity from WaSP droplet processing because there was insufficient signal intensity to observe adduct ions in similar proportion in the spectrum identified as FIG. 7A.

The value of developing a capLC/WaSP/MALDI target interface, becomes apparent when the typical analyte density achievable for different prior art sample preparation techniques are compared with those of WaSP. Analyte density is used because it has been predicted that the absolute detection limit in MALDI is dependent on the number of molecules/μm² occupying the sample spot targeted (5 molecules/μm² being the limit of detection). [59] Analyte density of a MALDI sample spot can be evaluated as,
analyte density=d=(C_{analyte}VNₐ)/tπr² eqn. 1
Where, C_{analyte}=the concentration of the analyte:matrix mixture, V=volume of mixture delivered to the target, Nₐ=Avogadro's number, t=thickness of the dried sample spot, and r=radius of the dried sample spot. Often, to compare two different preparations, the same starting solution would be used so C_{analyte} would be constant. For simplicity, it was assumed that the same thickness was achieved in both preparations. Thus, the two critical factors remaining would be the volume consumed and the resulting sample spot radius (eqn. 2).

$${d}_{1} = ( {C}_{analyte1} {V}_{1} {N}_{a} ) / {t}_{1} π {r}_{1}^{2} \\ {d}_{2} = ( {C}_{analyte2} {V}_{2} {N}_{a} ) / {t}_{2} π {r}_{2}^{2} \\ \begin{matrix}{d}_{1} / {d}_{2} = [ ( {C}_{analyte1} {V}_{1} {N}_{a} ) / {t}_{1} π {r}_{1}^{2} ] / [ ( {C}_{analyte2} {V}_{2} {N}_{a} ) / {t}_{2} π {r}_{2}^{2} ] \\ = {V}_{1} {r}_{2}^{2} / {V}_{2} {r}_{1}^{2}\end{matrix} \qquad eqn . 2$$

For example, dried droplets prepared by pipette delivering a 1.00 μL aliquot of the analyte:matrix mixture (CHCA was prepared at 10 mg/ml in 50:50 methanol:acetic acid and mixed 1:1 with 0.1% TFA in water) onto a stainless steel MALDI target resulted in sample spots ˜2.0 mm in diameter, an area of ˜3×10⁵ μm2 (see Table 1 below). A single sample spot prepared by delivering a single ˜300 pL droplet to the MALDI target by WaSP is typically 20 μm in diameter, corresponding to an area of ˜3×10² μm². If both spots were created from a solution that contained a peptide at 1 nM concentration, 6×10⁸ molecules would be in the pipette delivered spot and ˜2×10⁵ molecules would be in the WaSP sample spot. This corresponds to analyte densities of 200 and 575 molecules/μm² respectively. In this Example, the WaSP sample spot exhibits an analyte density of ˜3 times that observed for the pipette preparation and thus, based on this calculation, it appears that the sensitivity from a WaSP sample spot should be higher than the pipette delivered spot. Importantly, this would be achieved with 3333 times less volume and therefore less molecules consumed overall.

TABLE 1
Analyte densities for various MALDI sample preparations as calculated using equation 1.

| | Sample | Volume | Volume | Spot | Spot | Analyte | # of | |
|---|---|---|---|---|---|---|---|---|
| Sample | Volume | Consumed | Remaining | Radius | Area | Concentration | Molecules | Analyte Density |
| Preparation | (μL) | (μL) | (μL) | (μm) | (μm³) | (M) | In Spot | (molecules/μm²) |
| Dried droplet | 10.00 | 1.00 | 9 | 1000 | 314000 | 1 × 10⁻⁹ | 6.02 × 10⁸ | 192 |
| Hydrophobic Anchor | 10.00 | 10.00 | 0 | 1000 | 314000 | 1 × 10⁻⁹ | 6.02 × 10⁹ | 1917 |
| WaSP 1 droplet | 10.00 | 3 × 10⁻⁴ | 9.9997 | 10 | 314 | 1 × 10⁻⁹ | 1.81 × 10⁵ | 575 |
| WaSP 50 droplets | 10.00 | 0.015 | 9.985 | 50 | 7850 | 1 × 10⁻⁹ | 9.03 × 10⁶ | 1150 |
| WaSP 100 droplets | 10.00 | 0.030 | 9.970 | 50 | 7850 | 1 × 10⁻⁹ | 1.81 × 10⁷ | 2301 |
| Dried droplet | 10.00 | 1.00 | 9 | 1000 | 314000 | 8.7 × 10⁻¹² | 5.24 × 10⁶ | 2 |
| Hydrophobic Anchor | 10.00 | 10.00 | 0 | 1000 | 314000 | 8.7 × 10⁻¹² | 5.24 × 10⁷ | 17 |
| WaSP 1 droplet | 10.00 | 3 × 10⁻⁴ | 9.9997 | 10 | 314 | 8.7 × 10⁻¹² | 1.57 × 10³ | 5 |
| WaSP 50 droplets | 10.00 | 0.015 | 9.985 | 50 | 7850 | 8.7 × 10⁻¹² | 7.86 × 10⁴ | 10 |
| WaSP 100 droplets | 10.00 | 0.030 | 9.970 | 50 | 7850 | 8.7 × 10⁻¹² | 1.57 × 10⁵ | 20 |

Pipette delivery of sample aliquots to MALDI targets is by no means the only sample preparation method available. Table 1 summarizes similar calculations for increasing numbers of droplets prepared by WaSP as well as the use of a hydrophobic anchor. An ideal hydrophobic anchor would concentrate volumes as large as 10 μL as a spot ˜1000 μm in radius. This system would produce an analyte density of ˜1900 molecules/μm² using the solution discussed above, ˜3 times the calculated value for a single droplet from WaSP. However, this raises the issue of the sample loading capabilities available to WaSP, i.e. multiple droplets can be deposited onto the MALDI target in the same sample spot. This approach has been used for systems employing piezoceramic droplet dispensing devices to deliver sample material to a MALDI target. [60-62, 46, 63] If 100 droplets processed by WaSP can be delivered to create a sample spot with 100 μm diameter, an analyte density of ˜2300 molecules/μm² would be achieved. This would be slightly higher than the density achieved using the hydrophobic anchor while consuming only 30 nL, 333 times less than the 10 μL required using the hydrophobic anchors. If larger numbers of droplets could be concentrated into a similar area, even larger gains in analyte density could be achieved. This is an important result when the efficiency of MALDI is considered. It has been shown using radionuclides that when the analyte ion signal is depleted, ˜70% of the analyte still remains in the sample spot in a form that is not efficiently desorbed. [64] Considerable theoretical and experimental effort is being expended to address the fundamental issues in the MALDI process that will optimize matrix/ analyte co-crystallization and analyte ionization efficiency. [65-68] Gains achieved from this research will make MALDI sample preparation strategies, such as WaSP, that are capable of preparing μm-sized or smaller sample spots while consuming small volumes of liquid very advantageous.

The MALDI-MS data of this Example coincide with the predictions that WaSP can prepare sample spots with higher analyte density than the dried droplet method. For example, using equation 2 we can obtain an estimate of the analyte densities in the sample spots from which the spectra in FIG. 7 were generated. Here the concentration terms must be re-introduced because the fraction was manipulated when the matrix was added. Taking d₁ as the analyte density in the WaSP sample spot and d₂ as the analyte density in the dried droplet, the estimated increase in the analyte density for the WaSP sample spot was d₁/d₂=50. The intensity of the [T²²⁶-Y²⁴⁰+H⁺] ion in the WaSP sample spot was ˜5 times that measured from the dried droplet. It is likely that the ion intensity difference does not directly correlate with the analyte density difference of the sample because of the assumptions made in the density calculation. For instance, the thickness of the samples may differ or the eligible surface area of the crystals irradiated may not be uniform between the two preparations. Furthermore, the ionization efficiency for the different sample preparations are likely not identical and thus a term K is introduced into eqn. 2 to account for these factors when comparing the ion abundances produced from different MALDI sample spot preparations, resulting in eqn. 3.
d₁/d₂=K₁V₁r₂²/K₂V₂r₁² eqn. 3

Nonetheless, analyte ion density does affect the analyte ion signal measured so the prediction that small sample spots with sufficient analyte density may prove to be the most effective approach to achieving a decrease in the absolute detection limit in MALDI-MS should be further investigated. On that note, if a calculation is performed where the predicted analyte density of 5 molecules/μm² for the absolute limit of detection is used, an estimate of the lowest concentration of a peptide in a solution that can be prepared as a single 300 pl droplet by WaSP and still provide a useful ion abundance can be estimated as ˜9 pM (Table 1, italics). This would amount to a total consumption of 1570 molecules. If multiple droplet co-deposition is used, measurable ion current could be obtained from even lower concentration starting solutions. For example, if 100 droplets were deposited into the same size area created by the single droplet, a solution of 90 fM could be analyzed. A challenge in achieving this goal in practice has been the targeting of such small sample spots reproducibly using conventional MALDI MS instruments.

Sample spots created by WaSP processing of droplets from a fraction collected from the capLC separation of peptides produced by the proteolytic digestion of CCT produced higher analyte densities and improved peptide ion abundances relative to corresponding dried droplet and piezoelectric droplet dispenser preparations. This investigation of coupling capLC to MALDI using electrodynamic droplet processing has shown the feasibility of this strategy, primarily as a result of the pre-concentration initiated by solvent evaporation from the levitated droplets. A flow rate of ˜50 nl/min was accommodated while retaining the sample material spot sizes <200 μm in diameter. Implementation of other existing general strategies for coupling capLC to MALDI will increase the flow rate accommodated, such as heating the EDB chamber, employing a larger nozzle diameter on the droplet dispenser to increase the initial droplet volume, introducing a heated N₂ gas flow to lower the solvent vapor pressure and heating of the MALDI plate to increase the rate of solvent evaporation from the levitated droplets, as well as the use of MALDI target plates that are pre-coated with MALDI matrix. To accommodate very high capLC flow (i.e. >1 μL/min), the column eluent could also be split, or a droplet dispenser with a flow-through design could be utilized. [34] That is, depending upon the application, a flow regulator, such as a fractionator and/or flow splitter, could be employed between the upstream separator and the downstream droplet generator to improve matching of flow rates. The invention enables sample to be archived both on the MALDI plate and in the liquid form for subsequent complementary detection or manipulations. Furthermore, to expedite development, computerized control of the MALDI target plate translation and the potentials applied to the EDB electrodes, all synchronized to the droplet generation event, should be implemented. Overall, these results suggest that the WaSP methodology employed here may also be suitable for coupling with other low-flow separation technologies in similar analytical applications.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

### REFERENCES

1. Tanaka, K.; Waki, H.; Ido, Y.; Akita, S.; Yoshida, Y.; Yohida, T. Protein and polymer analyses up to m/z 100,000 by laser ionization time of flight mass spectrometry Rapid Commun. Mass Spectrom. 1988, 2, 151-153.

2. Karas, M.; Hillenkamp, F. Laser desorption ionization of proteins with molecular masses exceeding 10000 Daltons Anal. Chem. 1988, 60, 2299-2301.

3. Hillenkamp, F.; Karas, M.; Beavis, R.; Chait, B. Matrix Assisted laser desorption/ionization mass spectrometry of biopolymers Anal. Chem. 1991, 63, 1193A.

4. Shevchenko, A.; Sunyaev, S.; Loboda, A.; Shevchenko, A.; Bork, P.; Ens, W.; Standing, K. G. Charting the proteomes of organisms with unsequenced genomes by MALDI-quadrupole time-of-flight mass spectrometry and BLAST homology searching Anal. Chem. 2001, 73, 1917-1926.

5. Shevchenko, A.; Loboda, A.; Shevchenko, A.; Ens, W.; Standing, K. G. MALDI quadrupole time of flight mass spectrometry: A powerful tool for proteomic research Anal. Chem. 2000, 72, 2132-2141.

6. Amiri-Eliasi, B.; Fenselau, C. Characterization of protein biomarkers desrobed by MALDI from whole fungal cells Anal. Chem. 2001, 73, 5228-5231.

7. Berkenkamp, S.; Firpekar, F.; Hillenkamp, F. Infrared MALDI mass spectrometry of large nucleic acids Science 1998, 281, 260-262.

8. Gluckman, M.; Pfenninger, A.; Kruger, R.; Thierolf, M.; Karas, M.; Horneffer, V.; Hillenkamp, F.; Strupat, K. Mechanisms in MALDI analysis: surface interaction or incorporation of analytes Int. J. Mass Spectrom. 2001, 210/211, 121-132.

9. Zenobi, R.; Knochenmuss, R. Ion formation in MALDI mass spectrometry Mass Spectrom. Rev. 1998, 17, 337-366.

10. Knochenmuss, R.; Vertes, A. Time-delayed 2-pulse studeis of MALDI matrix ionization mechanisms J. Phys. Chem. B 2000, 104, 5406-5410.

11. Zhao, S.; Somayajula, K. V.; Sharkey, A. G.; Hercules, D. M.;Hillenkamp, F.; Karas, M.; Ingendoh, A. Anal. Chem. 1991, 63,450-453.

12. Chan, T.-W.; Colbum, A. W.; Derrick, P. J. Org. Mass Spectrom.1992, 27, 53-56.

13. Cornett, D. S.; Duncan, M. A.; Amster, I. J. Anal. Chem. 1993, 65, 2608-2613.

14. Zollner, P.; Stubiger, G.; Schmid, E.; Pittenauer, E.; Allmaier, G. Int. J. Mass Spectrom. 1997, 69/170, 99-109.

15. Williams, T. L.; Fenselau, C. Eur. Mass Spectrom. 1998, 4, 379-383.

16. Ring, S.; Rudich, Y. Rapid Commun. Mass Spectrom. 2000, 14, 515-519.

17. Sze, E. T. P.; Chan, T.-W. D.; Wang, G. J. Am. Soc. Mass Spectrom. 1997, 9, 166-174.

18. Yogi, O.; Kwawkami, T.; Yamauchi, M.; Ye, J. Y.; Ishikawa, M. Anal. Chem. 2001, 73, 1896-1902.

19. Johnson, T.; Bergquist, J.; Ekrnan, R.; Nordhoff, E.; Schurenberg, M.; Klöppel, K.-D.; Müller, M.; Lehrach, H.; Gobom, J. Anal. Chem. 2001, 73, 1670-1675.

20. Little, D. P.; Cornish, T. J.; O'Donnel, M. J.; Braun, A.; Cotter, R. J.; Koster, H. Anal. Chem. 1997, 69, 4540-4546.

21. Onnerfjord, P.; Nilsson, J.; Wallman, L.; Laurell, T.; Marko-Varga, G. Anal. Chem. 1998, 70, 4755-4760.

22. Jespersen, S. Rapid Commun. Mass Spectrom. 1994, 8, 581-584.

23. Schuerenberg, M.; Luebbert, C.; Eickhoff, H.; Kalkum, M.; Lehrach, H.; Nordhoff, E. Anal. Chem. 2000, 72, 3436-3442.

24. Keller, B. O.; Li, L. J. Am. Soc. Mass Spectrom. 2001, 12, 1055-1063.

25. Preisler, J.; Hu, P.; Rejtar, T.; Moskovets, E.; Karger, B. L. Anal. Chem. 2002, 74, 17-25.

26. Retjar, T.; Hu, P.; Juhasz, P.; Campbell, J. M.; Vestal, M. L.; Preisler, J.; Karger, B. L. J. Proteome Res. 2002, 1, 171-179.

27. Henion, J. D. J. Chromatogr. Sci 1980; 18: 101-2.

28. Henion, J. D. J. Chromatogr. Sci 1981; 19: 57-64.

29. Deterding, L. J., Moseley, M. A., Tomer, K. B., Jorgenson, J. W. J. Chromatogr 1991; 554: 73-82.

30. McLuckey, S., Van Berkel, G., Glish, G. L., Huang, E., Henion, J. Anal. Chem. 1991; 63: 375.

31. Murray, K. Mass Spectrom. Rev. 1997; 16: 283-299.

32. Li, J. J., Thibault, P., Bings, N. H., Skinner, C. D., Wang, C., Colyer, C., Harrison, J. Anal. Chem. 1999; 71: 3036-3045.

33. Creaser, C. S., Lill, J. R., Bonner, P. L. R., Hill, S. C., Rees, R. C. Analyst. Apr 2000; 125: 599-603.

34. Heiger-D N, S.-T. Anal. Chem. 2000; 72: 1236-1241.

35. Liu, J., Tseng, K., Garcia, B., Lebrilla, C. B., Mukerjee, E., Collins, S., Smith, R. Anal. Chem. 2001; 73: 2147-2151.

36. Wilm, M., Mann, M. Int. J. Mass Spectrom. Ion Processes 1994; 136: 167-180.

37. Valaskovic, G. A., Kelleher, N. L., Little, D., Aaserud, D., McLafferty, F. W. Anal. Chem. 1995; 67: 3802-3805.

38. Wilm, M., Mann, M. Anal. Chem. 1996; 68: 1-8.

39. Wilm, M. S., Neubauer, G., Mann, M. Anal. Chem. 1996; 68: 527-533.

40. Brivio, M., Fokkens, R. H., Verboom, W., Reinhoudt, D. N., Tas, N. R., Goedbloed, M., van den Berg, A. Anal. Chem. 2002; 74: 3972-3976.

41. Wall, D. B., Berger, S. J., Finch, J. W., Cohen, S. A., Richardson, K., Chapman, R., Drabble, D., Brown, J., Gostick, D. Electrophoresis 2002; 23: 3193-3204.

42. Zhang, B., McDonald, C., Li, L. Anal. Chem. 2004; 76: 992-1001.

43. Zhang, H., Caprioli, R. M. J. Mass Spectrom. 1996; 31: 1039-1046.

44. Zhang, H. Y., Stoekli, M., Andren, P. E., Caprioli, R. M. J. Mass Spectrom. 1999; 34: 377-383.

45. Milliotis, T., Kjellstrom, S., Nilsson, J., Laurell, T., Edholm, L. E., Marko-Varga, G. J. Mass Spectrom. 2000; 35: 369-377.

46. Ekstrom, S., Ericsson, D., Onnerfjord, P., Bengtsson, M., Nilsson, J., Marko-Varga, G., Laurell, T. Anal. Chem. 2001; 73: 214-219.

47. Hensel, R. R., King, R. C., Owens, K. G. Rapid Commun. Mass Spectrom. 1997; 11: 1785-1793.

48. Bogan, M. J., Agnes, G. R. Anal. Chem. 2002; 74: 489-496.

49. Bogan, M. J., Agnes, G. R. Rapid Commun. Mass Spectrom. 2003; 17: 2557-2562.

50. Davis, E. J., Buehler, M. F., Ward, T. L. Rev. Sci. Instrum. 1990; 61: 1281-1288.

51. Davis, E. J. Aerosol Sci. Tech. 1997; 26: 212-254.

52. Vehring, R., Aardahl, C. L., Davis, E. J., Schweiger, G., Covert, D. S. Rev. Sci. Instrum. 1997; 68: 70-78.

53. Zhu, J., Zheng, F., Laucks, M. L., Davis, E. J. J. Colloid Int. Sci. 2002; 249: 351-358.

54. Cornell, R. B., Northwood, I. C. Trends Biochem. Sci. 2000; 25: 441-447.

55. Friesen, J. A., Campbell, H. A., Kent, C. J. Biol. Chem. 1999; 274: 13384-13389.

56. Davies, S. M. A., Epand, R. M., Kraayenhof, R., Cornell, R. B. Biochemistry 2001; 40: 10522-10531.

57. Craig, L., Johnson, J. E., Cornell, R. B. J. Biol. Chem. 1994; 269: 3311-3317.

58. Bogan, M. J., Agnes, G. R. J. Am. Soc. Mass Spectrom. 2004; 15: 486-495.

59. Keller, B. O., Li, L. J. Am. Soc. Mass Spectrom. 2001; 12: 1055-1063.

60. Onnerfjord, P., Nilsson, J., Wallman, L., Laurell, T., Marko-Varga, G. Anal. Chem. 1998; 70: 4755-4760.

61. Onnerfjord, P., Ekstrom, S., Bergquist, J., Nilsson, J., Laurell, T., Marko-Varga, G. Rapid Commun. Mass Spectrom. 1999; 13: 315-322.

62. Ekstrom, S., Onnerfjord, P., Nilsson, J., Bengtsson, M., Laurell, T., Marko-Varga, G. Anal. Chem. 2000; 72: 286-293.

63. Miliotis, T., Marko-Varga, G., Nilsson, J., Laurell, T. J. Neurosci. Meth. 2001; 109: 4146.

64. Page, J. S., Sweedler, J. V. Anal. Chem. 2002; 74: 6200-6204.

65. Knochenmuss, R., Stortelder, A., Breuker, K., Zenobi, R. J. Mass Spectrom. 2000; 35: 1237-1245.

66. Knochenmuss, R., Zenobi, R. Chem. Rev. 2002; 1032: 441452.

67. Dreisewerd, K. Chem. Rev. 2003; 103: 395425.

68. Knochenmuss, R. Anal. Chem. 2003; 75: 2199-2207.

What is claimed is:

1. A method of preparing samples for use in analyte detection comprising:
(a) providing a supply of analyte;
(b) forming a test solution comprising said analyte and at least one volatile solvent;
(c) generating a discrete droplet of said test solution;
(d) electrodynamically levitating said droplet to enable evaporation of said volatile solvent, thereby increasing the concentration of said analyte in said droplet; and
(e) controllably depositing said droplet at a target location on a substrate to create at least one microspot thereon.

2. The method as defined in claim 1, comprising repeating steps (c)-(e) to successively deposit multiple droplets at said target location, thereby increasing the density of said analyte in said microspot.

3. The method as defined in claim 2, wherein said steps (c)-(e) are repeated sufficient times such that the density of said analyte in said microspot exceeds the minimum density detectable using MALDI-TOF mass spectrometry.

4. The method as defined in claim 3, wherein greater than 50 droplets are deposited on said microspot.

5. The method as defined in claim 4, wherein greater than 100 droplets are deposited on said microspot.

6. The method as defined in claim 1, wherein multiple droplets are deposited on said substrate at different locations to form multiple microspots thereon.

7. The method as described in claim 3, wherein said test solution comprises a MALDI matrix and wherein said substrate is a MALDI plate.

8. The method as defined in claim 3, wherein said electrodynamically levitating is performed by levitating said droplets in an electrodynamic balance.

9. The method as defined in claim 3, wherein said droplets follow an oscillatory flight path between said electrodynamic balance and said substrate.

10. The method as defined in claim 3, wherein said solution comprises a surface tension modifier to inhibit coulomb explosion of said droplet during said levitating.

11. The method as defined in claim 3, wherein said microspot is less than about 200 μm in diameter.

12. The method as defined in claim 11, wherein said sample spot is less than about 100 μm in diameter.

13. The method as defined in claim 1, wherein said analyte is a biomolecule.

14. The method as defined in claim 13, wherein said biomolecule is larger than about 500 Daltons in size.

15. The method as defined in claim 13, wherein said analyte is provided from a biomolecule separator.

16. The method as defined in claim 15, wherein said biomolecule separator is a capillary liquid chromatography column.

17. The method as defined in claim 16, wherein a supply of said analyte is received continuously from an outlet of said column.

18. The method as defined in claim 17, wherein the rate of generation of said droplets is synchronized with the flow rate of said analyte received from said column.

19. The method as defined in claim 16, wherein said electrodynamically levitating is performed by levitating said droplets in an electrodynamic balance and wherein said chromatography column is operatively coupled to said electrodynamic balance.

20. The method as defined in claim 2, wherein said forming of said test solution comprises mixing said analyte with a liquid MALDI matrix.

21. A method of detecting the presence of an analyte in a sample comprising:
(a) providing a supply of analyte;
(b) forming a test solution comprising said analyte and at least one volatile solvent;
(c) generating a discrete droplet of said test solution;
(d) electrodynamically levitating said droplet to enable evaporation of said volatile solvent, thereby increasing the concentration of said analyte in said droplet;
(e) controllably depositing said droplet at a target location on a substrate to create at least one microspot thereon and
(f) detecting the presence of said analyte in said microspot.

22. The method as defined in claim 21, wherein said detecting comprises irradiating said microspot and detecting ions produced by said irradiating by mass spectrometry.

23. The method as defined in claim 22, wherein said mass spectrometry is time of flight mass spectrometry.

24. The method as defined in claim 22, comprising, prior to said irradiating, repeating steps (c)-(e) to successively deposit multiple droplets at said target location, thereby increasing the density of said analyte in said microspot.

25. The method as defined in claim 24, wherein said steps (c)-(e) are repeated sufficient times such that the density of said analyte in said microspot exceeds the minimum density detectable using MALDI-TOF mass spectrometry.

26. The method as defined in claim 25, wherein said detecting comprises irradiating said microspot and detecting ions produced by said irradiating by MALDI-TOF mass spectrometry.

27. The method as defined in claim 24, wherein greater than 50 droplets are deposited on said microspot.

28. The method as defined in claim 27, wherein greater than 100 droplets are deposited on said microspot.

29. The method as defined in claim 24, wherein multiple droplets are deposited on said substrate at different locations to form multiple microspots thereon.

30. The method as described in claim 36, wherein test solution comprises a MALDI matrix and wherein said substrate is a MALDI plate.

31. The method as defined in claim 21, wherein said electrodynamically levitating is performed by levitating said droplets in an electrodynamic balance.

32. The method as defined in claim 31, wherein said droplets follow an oscillatory flight path between said electrodynamic balance and said substrate.

33. The method as defined in claim 21, wherein said solution comprises a surface tension modifier to inhibit coulomb explosion of said droplet during said levitating.

34. The method as defined in claim 21, wherein said microspot is less than about 200 μm in diameter.

35. The method as defined in claim 34, wherein said sample spot is less than about 100 μm in diameter.

36. The method as defined in claim 21, wherein said analyte is a biomolecule.

37. The method as defined in claim 36, wherein said biomolecule is larger than about 500 Daltons in size.

38. The method as defined in claim 36, wherein said analyte is provided from an upstream biomolecule separator.

39. The method as defined in claim 38, wherein said biomolecule separator is a capillary liquid chromatography column.

40. The method as defined in claim 39, wherein a supply of said analyte is received continuously from an outlet of said column.

41. The method as defined in claim 40, wherein the rate of droplet generation is synchronized with the flow rate of said analyte.

42. The method as defined in claim 39, wherein said electrodynamically levitating is performed by levitating said droplets in an electrodynamic balance and wherein said chromatography column is operatively coupled to said electrodynamic balance.

43. The method as defined in claim 21, wherein said matrix is a liquid matrix.

44. The method as defined in claim 43, wherein said liquid matrix is a MALDI matrix.

45. An analyte detection system comprising:
(a) an analyte supply;
(b) a vessel for forming a test solution comprising analyte received from said analyte supply and at least one volatile solvent;
(c) a droplet generator for generating discrete droplets of said solution; and
(d) an electrodynamic balance for electrodynamically levitating said droplets produced by said droplet generator for a sufficient length of time to enable evaporation of said volatile solvent and hence concentration of said analyte in said droplets, wherein said electrodynamic balance successively ejects droplets to a target location following levitation thereof.

46. The system as defined in claim 45, further comprising a laser for irradiating a sample location of said substrate.

47. The system as defined in claim 46, further comprising a MALDI-TOF mass spectrometer for detecting ions produced by said irradiating of said sample location.

48. The system as defined in claim 45, further comprising a substrate at said target location for receiving said droplets.

49. The system as defined in claim 48, wherein said test solution comprises a MALDI matrix and wherein said substrate is a MALDI plate.

50. The system as defined in claim 45, wherein said analyte supply is a biomolecule separator.

51. The system as defined in claim 50, wherein said biomolecule separator is a capillary liquid chromatography column.

52. The system as defined in claim 51, wherein said analyte is supplied continuously from said column to said vessel.

53. The systems as defined in claim 45, wherein said solution comprises a surface tension modifier.

54. The system as defined in claim 53, wherein said modifier is glycerol.

55. The system as defined in claim 51, wherein said column is fluidly coupled to said vessel.

56. The system as defined in claim 45, further comprises a flow regulator for regulating the rate of flow of analyte from said analyte supply, wherein the regulated flow rate of analyte matches the rate of downstream droplet generation.

57. A method of preparing samples for use in analyte detection comprising:
(a) providing a supply of analyte;
(b) forming a test solution comprising said analyte and at least one volatile solvent;
(c) generating a discrete droplet of said test solution;
(d) electrodynamically levitating said droplet to enable evaporation of said volatile solvent, thereby increasing the concentration of said analyte in said droplet; and
(e) controllably ejecting said droplet to a target location following levitation thereof.

58. The method as defined in claim 57, wherein said target location is a substrate and said droplets form one or more microspots on said substrate.

59. The method as defined in claim 57, wherein said target location is the input orifice of a mass spectrometer.
